(19) United States
(12) Patent Application Publication
(10) Pub. No.: US 2026/0237130 A1
(43) Pub. Date: Aug. 13, 2026

(54) COMPUTER-IMPLEMENTED METHOD FOR ASCERTAINING A SCATTERED RADIATION CONTRIBUTION TO AN EVALUATION IMAGE, EVALUATION FACILITY, COMPUTER PROGRAM AND ELECTRONICALLY READABLE DATA CARRIER

(71) Applicant: Siemens Healthineers AG, Forchheim (DE)

(72) Inventor: Philipp Roser, Allston, MA (US)

(21) Appl. No.: 19/539,653

(22) Filed: Feb. 13, 2026

(30) Foreign Application Priority Data
Feb. 13, 2025 (DE) 10 2025 105 314.6

(51) Int. Cl. G06T 12/30; G06N 3/0464; G06N 3/08; G06V 10/74; G16H 30/20

(52) U.S. Cl. CPC G06T 12/30; G06N 3/0464; G06N 3/08; G06V 10/761; G16H 30/20

(57) ABSTRACT

A method for ascertaining a scattered radiation contribution to an evaluation image recorded with an X-ray facility includes supplying an encoder of a trained ascertainment function. The method includes supplying an evaluation database in which reference datasets are stored that, for each training dataset with which the ascertainment function was trained, include: the training scattered radiation image of the training dataset specifying the basic truth; and the feature vector for the training dataset. The method includes applying the encoder to the evaluation image to ascertain a feature vector of the evaluation image, comparing the feature vector of the evaluation image with the feature vectors of the reference datasets to ascertain at least one reference dataset with the most similar feature vector from the evaluation database, and ascertaining a scattered radiation image for the evaluation image from the training scattered radiation images of the at least one ascertained reference dataset.

This application claims the benefit of German Patent Application No. DE 10 2025 105 314.6, filed on Feb. 13, 2025, which is hereby incorporated by reference in its entirety.

### BACKGROUND

The present embodiments relate to a computer-implemented method and an evaluation facility for ascertaining a scattered radiation contribution to an evaluation image recorded with an X-ray facility, to a computer program, and an electronically readable data carrier.

In X-ray imaging, it is possible for scattered radiation to occur (e.g., due to scatter processes at the object to be recorded and/or other radiographed objects), and this may also strike the X-ray detector of the corresponding X-ray facility and is measured accordingly. Such scattered radiation contributions may be undesirable since scattered radiation contributions may make evaluation of the corresponding X-ray image difficult. Scattered radiation has a particularly negative influence in cone beam computed tomography (CBCT) in which projection images are recorded in different directions of projection with a recording arrangement (e.g., a C-arm X-ray facility), from which images a three-dimensional X-ray image is then reconstructed. Without suitable compensation, the scattered radiation may result in drastic losses in the image quality of the reconstructed image dataset (e.g., in stripe artifacts, cupping artifacts, and blurring artifacts). This may result in an impeded or even erroneous evaluation.

It is therefore known in the prior art to arrange an anti-scatter grid in front of the X-ray detector on X-ray facilities. The grid is intended to reduce the incident X-ray radiation as far as possible to X-rays directly incident from the X-ray tube assembly by physically blocking X-ray beams from other directions. However, such anti-scatter grids also have adverse effects.

First, the anti-scatter grid inevitably also blocks some of the primary radiation, so an increase in the X-ray dose may potentially be necessary. Further, improved image quality and a lower dose may be achieved in two-dimensional X-ray imaging (e.g., therefore radiography) by removing the anti-scatter grid in that what is known as the Air Gap technique is used in which the X-ray detector is brought as close as possible to the object to be recorded. This applies, for example, to neurovascular imaging of the brain (e.g., when examining aneurysms and in the case of embolic strokes). To be able to supply X-ray facilities without anti-scatter grid, which are also intended for use in high-quality two-dimensional imaging (e.g., of the brain), a software solution is necessary for the scattered radiation correction, at least in the case of three-dimensional imaging procedures (e.g., in cone beam computed tomography).

Approaches have already been proposed in the prior art, in which machine learning is used to infer scattered radiation distributions from X-ray images. However, these approaches may be less robust since outliers, for example, may occur when estimating the scattered radiation. It is known to ascertain scattered radiation distributions via simulation of the imaging procedure, although greater computing effort and greater calculation time are required for this.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a fast and robust possibility of ascertaining high-quality items of scattered radiation information in an X-ray facility is provided.

In one embodiment, a method for ascertaining a scattered radiation contribution to an evaluation image recorded with an X-ray facility has the following acts: supplying at least one encoder of a trained ascertainment function, which includes the encoder, which supplies a feature vector in a latent space for an X-ray image supplied as input data, and a decoder, which ascertains an output image, describing the scattered radiation contribution in the X-ray image, from the feature vector; supplying an evaluation database in which reference datasets are stored that, for each training dataset with which the ascertainment function was trained, in each case include at least: the training scattered radiation image of the training dataset specifying the basic truth; and the feature vector for the training dataset; applying the encoder to the evaluation image in order to ascertain a feature vector of the evaluation image; comparing the feature vector of the evaluation image with the feature vectors of the reference datasets in order to ascertain at least one reference dataset with the most similar feature vector from the evaluation database; and ascertaining a scattered radiation image for the evaluation image from the training scattered radiation images of the at least one ascertained reference dataset.

Inventively, it is therefore provided to train an embedding encoder using machine learning in order to map evaluation images (e.g., X-ray images to be evaluated) that are, for example, projection images of a three-dimensional imaging procedure (e.g., from cone beam computed tomography) onto a feature vector (e.g., embedding vector) that lies in a latent space. In addition, an evaluation database is supplied that may also be regarded as an atlas or a Look-Up Table (LUT). The evaluation database includes entries, which in each case include at least feature vectors that, on application of the encoder to training X-ray images, pertaining to training datasets for training, with the respectively assigned training scattered radiation images (e.g., the basic truth and form the reference datasets). This is possible because the encoder is not trained in isolation, but rather together with a decoder that delivers output images describing the scattered radiation distribution. The encoder and the decoder form the ascertainment function, which may be trained by training datasets that contain training X-ray images and training scattered radiation images.

After training, only the encoder is used to ascertain feature vectors for all training datasets (e.g., all data pairs). The feature vector and the respective training scattered radiation image of the training dataset are stored in the evaluation database as reference datasets. Translated, the feature vector may be understood as a kind of hash key while the assigned data (e.g., at least the training scattered radiation image) may be understood as stored values in a k value map.

During an image-recording procedure, at least one two-dimensional X-ray image to be evaluated (e.g., projection image) is then recorded as an evaluation image. The evaluation image is transferred as input data to the encoder in order to determine a feature vector. The feature vector is then used as a query for the evaluation database. At least the reference dataset having a feature vector that is most similar (e.g., closest) to the feature vector of the evaluation image is ascertained in the evaluation database and therefore retrieved. The scattered radiation image for the evaluation image is then ascertained from the training scattered radiation image of the at least one ascertained reference dataset. In other words, suitable predetermined scattered radiation images that reproduce a basic truth are found in the evaluation database and used to ascertain the most suitable scattered radiation image for the current evaluation image.

Fast, efficient and robust ascertainment of scattered radiation distributions for specific X-ray images to be evaluated that may be used, for example, for correction thereof, is supplied thereby. Therefore, the scattered radiation image may be used for scattered radiation correction of the evaluation image. Because only the encoder, but not the decoder, is now used, the runtime of the ascertainment is shortened. Further, false estimations of the decoder are prevented, so the robustness increases.

In general, a trained function maps cognitive functions that humans associate with other human brains. By training based on training data (e.g., machine learning), the trained function is capable of adapting to new circumstances and of detecting and extrapolating patterns. Another expression for “trained function” is “trained machine learning model.”

Generally speaking, parameters of a trained function may be adjusted via training. For example, supervised learning, semi-supervised learning, unsupervised learning, reinforcement learning, and/or active learning may be used. Further, representative learning (also known as “feature learning”) may also be used. The parameters of the trained function may, for example, be iteratively adjusted by a plurality of training steps. For example, a specific cost function may be minimized during training. For example, the backpropagation algorithm may be used when training a neural network.

A trained function may include, for example, a neural network, a Support Vector Machine (SVM), a decision tree and/or a Bayesian network, and/or the trained function may be based on k-means clustering, Q-Learning, genetic algorithms, and/or association rules. For example, a neural network may be a deep neural network, a Convolutional Neural Network (CNN), or a deep CNN. Further, the neural network may be an adversarial network, a deep adversarial network, and/or a Generative Adversarial Network (GAN).

A convolutional neural network (CNN) is a neural network that uses a convolutional operation instead of general matrix multiplication in at least one of its layers, what is known as the convolutional layer. For example, a convolutional layer may carry out a scalar product of one or more convolutional kernel(s) with the incoming data/images of the convolutional layer, with the entries of the one or more convolutional kernel(s) being the parameters or weights which are adjusted via training. For example, the Frobenius inner product and the ReLu activation function may be used. A CNN may include additional layers (e.g., pooling layers, fully connected layers, and normalization layers).

Input images may be processed extremely efficiently by CNN since a convolutional operation based on different kernels may extract a wide variety of image features, so, by adapting the weights of the convolutional kernel, the relevant image features may be found during training. Further, based on sharing of the weights in the convolutional layer kernels, fewer parameters are to be trained, so an overfitting is prevented in the training phase, and faster training or a greater number of layers is allowed in the CNN, so the performance of the network is increased. In this respect, the use of at least one CNN for the trained ascertainment function (e.g., the encoder and/or the decoder) may also be provided in the framework of this disclosure.

The trained ascertainment function may include a U-Net and/or an autoencoder (e.g., a variational autoencoder) and/or a transformer network. The U-Net, described, for example, in O. Ronneberger et al., “U-Net: Convolutional Networks for Biomedical Image Segmentation,” arXiv:1505.04597v1, includes a contracting path (e.g., the encoder) and an expanding path (e.g., the decoder) in a U-shaped architecture. The contracting path is a typical convolutional network with different convolutions, with a ReLU layer and a max-pooling layer following each one. During the contraction, the spatial information is reduced while feature information is increased. The expanding path combines the items of feature information and the items of spatial information via a sequence of up-convolutions and concatenations with high-resolution features of the contracting path. In a variational autoencoder, the encoder maps each point of a large complex dataset, instead of in a single point in the latent space, onto a distribution in the latent space, with the decoder accordingly mapping back into the input data space (e.g., the image space), likewise based on the distribution in the latent space.

In one embodiment, it may be provided that the method includes training the trained ascertainment function, where the following is provided for joint training of the encoder and decoder: supplying training datasets that in each case include a training X-ray image and an associated training scattered radiation image used as a basic truth, and the ascertainment function to be trained; training the ascertainment function using the training datasets; storing the feature vector for each training dataset; and ascertaining the reference datasets from the training datasets and the assigned feature vectors based on the trained ascertainment function.

The reference datasets then form the evaluation database and may be supplied similarly to the trained ascertainment function or its trained encoder.

Specifically, it may be provided that the training scattered radiation images are ascertained, at least partially (e.g., completely) by simulation. While it is basically also conceivable to determine scattered radiation images via measuring in, albeit laborious, measuring procedures (e.g., on phantoms), simulated X-ray scattered radiation images may be used, for example, as training scattered radiation images. The training X-ray images may also be the result of a simulation. However, it may also be provided that the training X-ray images are ascertained at least partially via a recording procedure, with image-recording parameters of the recording procedure also being used for simulation of an associated training scattered radiation image. Appropriate simulation methods are already largely known in the prior art and may also be used accordingly in the framework of this disclosure for ascertaining training data.

In a development of the present embodiments, it is provided that in the training, apart from a loss function of the supervised learning, which assesses the conformity of the output image with the training scattered radiation image, a further loss function of the unsupervised learning is used, which assesses the similarity of the feature vectors with respect to the similarity of the training scattered radiation images and/or output images. In other words, this provides that contrastive learning is used as the unsupervised learning. As it makes use of similarity and dissimilarity, contrastive learning machine learning models enable very similar instances to be mapped close to one another in the latent space, while dissimilar instances are spaced apart from one another in the latent space.

However, in the present case, in contrast to “conventional” contrastive learning, first, the focus will be on the similarity of the scattered radiation information, and not the input data second, the contrastive learning will also be effected, so reference may be made to semi-supervised learning that is aimed not only at an ideal conformity of the output images with the training scattered radiation images, but also selects the latent space representation (e.g., the construction of the feature vectors), such that a consistent (e.g., “smooth”) sorting also exists according to the similarity of the scattered radiation distribution. In other words, for more similar scattered radiation distributions, a more similar representation should also exist in the latent space, while for less similar items of scattered radiation information, feature vectors that are further apart should also exist. The focus is therefore on the output images of the decoder and/or the training scattered radiation images, and not on the training input images. With regard to the assignment, provided in the application, to reference datasets using the similarity of the feature vectors, the merging of similar scattered radiation images also results in the latent space to a much more robust and accurate estimation of the scattered radiation contributions.

Specifically, the feature vector is therefore also exported as an auxiliary output during training and may enter the further loss function accordingly. In example embodiments, it may be provided that a contrastive loss and/or a triplet loss is used as a further loss function. Both types of loss function are common in contrastive learning and also lead to advantageous results in the framework of the present embodiments. The further loss function may use a measure of distance and/or a measure of similarity between feature vectors and/or a corresponding measure between scattered radiation images.

Generally speaking, it is therefore provided that the ascertainment function is trained using a supervised loss with regard to the basic truth (e.g., training scattered radiation images) and using an unsupervised loss with respect to the distribution of feature vectors, with the corresponding further loss function focusing on similar training scattered radiation images also being close to one another in the latent space of the feature vectors. Put another way, the feature vector supplies a representation (e.g., embedding) that optimally maps the similarities and differences between the features of the data (e.g., the scattered radiation distributions).

In general, a development may provide that a plurality of reference datasets is ascertained, where the scattered radiation image is ascertained from a combination of the training scattered radiation images of the ascertained reference datasets and/or a combination of intermediate images derived therefrom. In one embodiment, not only the reference dataset of the closest feature vector may be used, but a plurality of reference datasets may be ascertained based on a corresponding measurement of distance and/or similarity of the feature vectors. For example, the training scattered radiation images and/or intermediate images may be linearly combined in order to ascertain the scattered radiation image for the evaluation image. It may be provided that the ascertained reference datasets are incorporated in the combination in a weighted manner with a distance measurement of the feature vector of the evaluation image for the feature vector of the respective reference dataset. However, the feature vector of the evaluation image may be represented as a linear combination of the feature vectors of the ascertained reference datasets, with the coefficients of the linear combination being incorporated as weights in the combination of the training scattered radiation images and/or intermediate images.

In one embodiment, the training X-ray image may also be stored in the reference datasets, where when ascertaining the scattered radiation image for the evaluation image, an item of deviation information of the evaluation image from the training X-ray image of the at least one ascertained reference dataset is used. In this way, possibly meaningful modifications to the training scattered radiation image of the at least one ascertained reference dataset may therefore be derived and applied in order to further increase the quality of the scattered radiation image ascertained for the evaluation image. In the process, differences from the corresponding training X-ray image that possibly exist are determined as an item of deviation information and taken into account when ascertaining the scattered radiation image for the evaluation image.

Specifically, it may be provided, for example, that the item of deviation information includes a ratio of the (e.g., mean or added) intensities of the evaluation image and the training X-ray image, with which the training scattered radiation image is scaled. Optionally, the training scattered radiation image retrieved from the evaluation database may therefore be scaled by the ratio of the intensity of the evaluation image to the intensity of the retrieved training X-ray image. A significant further improvement in the quality of the scattered radiation estimation is thus already achieved.

In addition or alternatively, it may be provided that the item of deviation information includes a transformation that the evaluation image links to the training X-ray image and/or is ascertained from the feature vectors of the evaluation image and the at least one ascertained reference dataset. The transformation is applied to the training scattered radiation image. Specifically, the transformation may include a deformation field, based, for example, on spline functions (e.g., B-splines) and/or a trained transformation function. In one embodiment, a more complex low-frequency transformation may be calculated based on the retrieved training X-ray image of the reference dataset and the recorded X-ray image to be evaluated (e.g., the evaluation image). This transformation may be based, for example, on a, for example, spline-based deformation field and/or an implicit trained transformation function (e.g., an implicit neural network). For example, known techniques for registering images may be used for this. The transformation is then applied to the retrieved training scattered radiation image of the ascertained reference dataset in order to adjust the scattered radiation distribution to the evaluation image. In an alternative embodiment, it may also be provided that the transformation is calculated based on the feature vectors. In such a case, the use of artificial intelligence (e.g., a trained transformation function) may be provided. If it is possible to trace and assign the features described by the feature vector, then it is possible to also use an analytical transformation function parameterized by the feature vectors and/or the differences thereof.

Basically, in the framework of the present embodiments, the decoder of the trained ascertainment function may be further utilized (e.g., in less time-critical applications). Then, it may be provided, for example, that the decoder of the trained ascertainment function is also supplied and used, where an output image, ascertained with the decoder, for the evaluation image and the scattered radiation image for the evaluation image are used for a reciprocal plausibility check. In this case, the scattered radiation image for the evaluation image, which was ascertained with the aid of the evaluation database, may be used for the plausibility check of the current output image for the evaluation image since it was ascertained more robustly. It is thus possible to quickly identify incorrect calculations of the decoder.

Apart from the method, the present embodiments also relate to an evaluation facility for ascertaining a scattered radiation contribution to an evaluation image recorded with an X-ray facility, having a storage device in which the following are stored: at least one encoder of a trained ascertainment function that includes the encoder. that supplies a feature vector in a latent space for an X-ray image provided as input data, and a decoder that ascertains an output data image describing the scattered radiation contribution in the X-ray image, from the feature vector; and an evaluation database in which reference datasets are stored that, for each training dataset with which the ascertainment function was trained, in each case include at least: the training scattered radiation image of the training dataset specifying the basic truth; and the feature vector for the training dataset. The evaluation facility also has: an application unit for applying the encoder to the evaluation image in order to ascertain a feature vector of the evaluation image; a first ascertainment unit for comparing the feature vector of the evaluation image with the feature vectors of the reference datasets and in order to ascertain at least one reference dataset with the most similar feature vector from the evaluation database; and a second ascertainment unit in order to ascertain a scattered radiation image for the evaluation image from the training scattered radiation images of the at least one ascertained reference dataset.

All statements with respect to the method of the present embodiments may be transferred analogously to the evaluation facility of the present embodiments, and vice versa, so the advantages that have already been mentioned may also be obtained with the evaluation facility.

The evaluation facility has at least one processor and the storage device. Hardware and/or software form functional units that allow acts of the method of the present embodiments to be carried out. Apart from the application unit and the first or second ascertainment unit, these may also include further functional units (e.g., a training unit for training the ascertainment function and/or a correction unit for applying the scattered radiation image to the evaluation image for correction of the evaluation image).

The evaluation facility may be provided as part of an imaging X-ray facility (e.g., integrated in its control facility, such as in order to be able to correct recorded X-ray images as evaluation images with regard to the scattered radiation directly, such as in the framework of a three-dimensional imaging procedure). For example, the evaluation image may be a projection image of a cone beam computed tomography recording. If each projection image is now corrected with regard to the scattered radiation, it is possible thereafter to reconstruct a three-dimensional image dataset of improved quality (e.g., a reduced number of artifacts).

An X-ray facility that includes an evaluation facility of the present embodiments may therefore be provided. The X-ray facility also includes an X-ray tube assembly and an X-ray detector as well as a control facility. An evaluation image recorded with the X-ray facility may be evaluated, and, for example, corrected, by the evaluation facility in order to ascertain the associated scattered radiation image. The X-ray facility may be, for example, an X-ray facility with a C-arm on which the X-ray tube assembly and the X-ray detector are arranged opposite one another. X-ray facilities of this kind are frequently used in angiography and/or for monitoring (e.g., minimally invasive) medical interventions. For example, the X-ray facility may be used for neurovascular imaging. Diagnostic X-ray images and/or those recorded alongside an intervention (e.g., projection images of a cone beam computed tomography) may be corrected of scattered radiation contributions directly in situ before being displayed on a display facility and/or being used to reconstruct a higher-dimensional image dataset.

A computer program of the present embodiments may be loaded directly into a storage device of an evaluation facility and has program means in such a way that when the computer program is executed on the evaluation facility, the evaluation facility is prompted to carry out a method of the present embodiments. The computer program may be stored on an electronically readable data carrier of the present embodiments, which therefore includes items of control information stored thereon. The items of control information include at least one computer program of the present embodiments and are configured such that when the data carrier is used in an evaluation facility, the facility is configured to carry out a method of the present embodiments. The data carrier may be, for example, a non-transient data carrier (e.g., a CD-ROM).

### BRIEF DESCRIPTION OF THE DRAWINGS

Further advantages and details of the present invention may be found in the example embodiments described below, as well as based on the drawings, in which:

FIG. 1 shows an example embodiment of a neural network;

FIG. 2 shows an example embodiment of a convolutional neural network;

FIG. 3 shows a schematic representation for training an ascertainment function;

FIG. 4 shows a schematic illustration of an evaluation database;

FIG. 5 schematically shows sequences and correlations in a first example embodiment of a method;

FIG. 6 schematically shows sequences and correlations in a second example embodiment of the method;

FIG. 7 shows a functional schematic diagram of an embodiment of an evaluation facility; and

FIG. 8 shows a schematic diagram of an embodiment of an X-ray facility.

### DETAILED DESCRIPTION

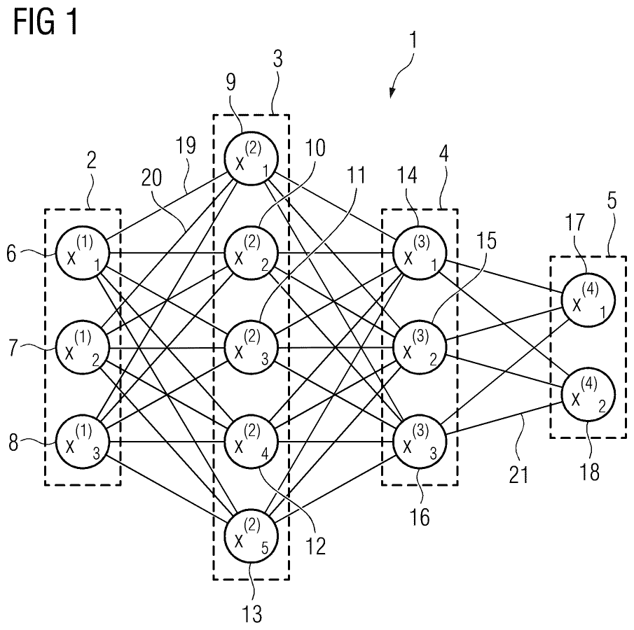

FIG. 1 shows an example embodiment of an artificial neural network 1. English expressions for the artificial neural network 1 are “artificial neural network,” “neural network,” “artificial neural net,” or “neural net.”

The artificial neural network 1 includes nodes 6 to 18 (e.g., nodes) and edges 19 to 21 (e.g., edges), with each edge 19 to 21 being a directed connection from a first node 6 to 18 to a second node 6 to 18. In general, the first nodes 6 to 18 and the second nodes 6 to 18 are different nodes 6 to 18, although the first node 6 to 18 and the second node 6 to 18 may be identical. For example, in FIG. 1, the edge 19 is a directed connection from the node 6 to the node 9, and the edge 21 is a directed connection from the node 16 to the node 18. An edge 19 to 21 from a first node 6 to 18 to a second node 6 to 18 is referred to as ingoing edge for the second node 6 to 18 and as an outgoing edge for the first node 6 to 18.

In this example embodiment, the nodes 6 to 18 of the artificial neural network 1 are arranged in layers 2 to 5 (e.g., layers), with it being possible for the layers to have an intrinsic order, which is introduced between the nodes 6 to 18 by the edges 19 to 21. For example, edges 19 to 21 may be provided only between adjacent layers of nodes 6 to 18. In the represented example embodiment, an input layer 2 that has solely the nodes 6, 7, 8, in each case without ingoing edge, exists. The output layer 5 includes only the nodes 17, 18, in each case without outgoing edges, with hidden layers 3 and 4 also being located between the input layer 2 and the output layer 5. In the general case, the number of hidden layers 3, 4 may be arbitrarily selected. The number of nodes 6, 7, 8 of the input layer 2 customarily corresponds to the number of input values in the neural network 1, and the number of nodes 17, 18 in the output layer 5 customarily corresponds to the number of output values of the neural network 1.

For example, a (real) number may be assigned to the nodes 6 to 18 of the neural network 1. In this case, x⁽ⁿ⁾ᵢ denotes the value of the i^{th} node 6 to 18 of the n^{th} layer 2 to 5. The values of the nodes 6, 7, 8 of the input layer 2 are equivalent to the input values of the neural network 1, while the values of the nodes 17, 18 of the output layer 5 are equivalent to the output values of the neural network 1. Further, each edge 19, 20, 21 may be assigned a weight in the form or a real number. For example, the weight is a real number in the interval [−1, 1] or in the interval [0, 1,]. In this case, w^{(m,n)}_{i,j} denotes the weight of the edge between the i^{th} nodes 6 to 18 of the m^{th} layer 2 to 5 and the j^{th} nodes 6 to 18 of the n^{th} layer 2 to 5. Further, the abbreviation

$${w}_{i , j}^{( n )}$$

is defined for the weight

$${w}_{i , j}^{( n , n + 1 )} .$$

To calculate output values of the neural network 1, the input values are propagated by the neural network 1. For example, the values of the nodes 6 to 18 of the (n+1)^{th} layer 2 to 5 may be calculated based on the values of the nodes 6 to 18 of the n^{th} layer 2 to 5 by

$${x}_{j}^{( n + 1 )} = f ( {\sum }_{i} {x}_{i}^{( n )} {w}_{i , j}^{( n )} ) .$$

In this case, f is a transfer function, which may also be referred to as an activation function. Known transfer functions are step functions, sigmoid functions (e.g., the logistical function, the generalized logistical function, the hyperbolic tangent, the arc tangent, the error function, the smoothstep function) or rectifier functions (e.g., rectifier). The transfer function is substantially used for standardization purposes.

For example, the values are propagated layer by layer through the neural network 1, with values of the input layer 2 being provided by the input data of the neural network 1. Values of the first hidden layer 3 may be calculated based on the values of the input layer 2 of the neural network 1, values of the second hidden layer 4 may be calculated based on the values in the first hidden layer 3, etc.

To be able to stipulate the values

$${w}_{i , j}^{( n )}$$

for the edges 19 to 21, the neural network 1 is to be trained using training data. For example, training data includes training input data and training output data, which will hereinafter be referred to as tᵢ. For a training step, the neural network 1 is applied to the training input data in order to ascertain calculated output data. For example, the training output data and the calculated output data include a number of values, with the number being determined as the number of nodes 17, 18 of the output layer 5.

For example, a comparison between the calculated output data and the training output data is used to recursively adjust the weights within the neural network 1 (e.g., back propagation algorithm). For example, the weights may be altered according to

$${w}_{i , j}^{′ ( n )} = {w}_{i , j}^{( n )} - γ \cdot {δ}_{j}^{( n )} \cdot {x}_{i}^{( n )}$$

where γ is a learning rate and the numbers δⱼ⁽ⁿ⁾ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on

$${δ}_{j}^{( n + 1 )}$$

if the (n+1)^{th} layer is not the output layer 5, and

$${δ}_{j}^{( n )} = ( {x}_{j}^{( n + 1 )} \cdot {t}_{j}^{( n + 1 )} ) \cdot {f}^{′} ( {\sum }_{i} {x}_{i}^{( n )} \cdot {w}_{i , j}^{( n )} )$$

if the (n+1)^{th} layer is the output layer 5, where f′ is the first derivative of the activation function, and

$${t}_{j}^{( n + 1 )}$$

is the comparison training value for the j^{th} node 17, 18 of the output layer 5.

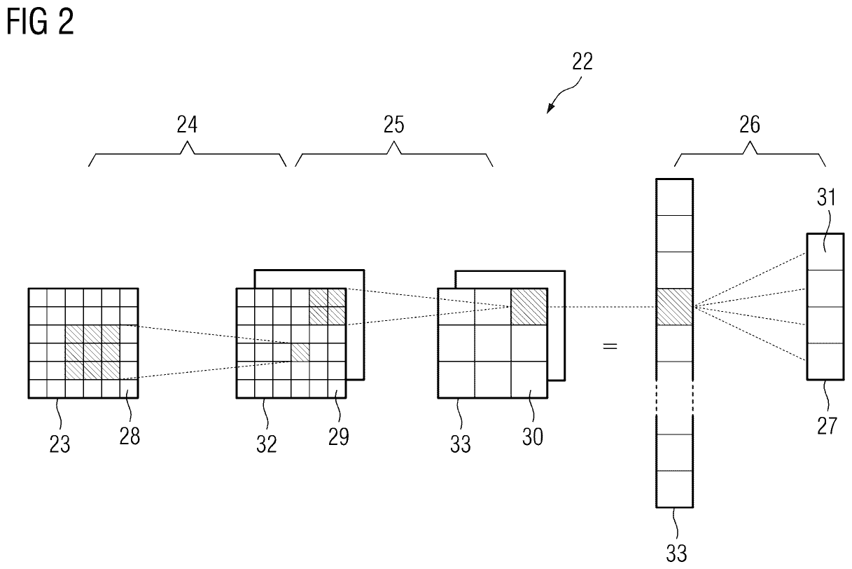

An example of a convolutional neural network (CNN) will also be provided hereinafter with regard to FIG. 2. It should be noted in this connection that the expression “layer” is often read in two different ways there. The expression “layer” refers to the set of nodes that is connected to other groups of nodes of this kind via ingoing and/or outgoing edges. The expression “layer” may be understood as two groups of nodes that are connected via a specific structure of edges (e.g., convolutional layer, pooling layer, fully connected layer), so each group of nodes is assigned to two such “layers”. For differentiation, the first type of layers is referred to as node layers, and the second type of layers (e.g., if it is not clear in some other way from the name) is referred to as connecting layers.

FIG. 2 shows an example embodiment of a convolutional neural network 22. In the represented example embodiment, the convolutional neural network 22 includes an input node layer 23, a convolutional layer 24, a pooling layer 25, a fully connected layer 26, and an output node layer 27 as well as hidden node layers 32, 33. In alternative embodiments, the convolutional neural network 22 may include a plurality of convolutional layers 24, a plurality of pooling layers 25, and a plurality of fully connected layers 26, as well as other types of layers. The order of the layers may be arbitrarily selected, with fully connected layers 26 customarily forming the last layers before the output layer 27.

For example, within a convolutional neural network 22, the nodes 28 to 30 of one of the node layers 23, 32 and 33 may be understood as being arranged in a d-dimensional matrix or as a d-dimensional image. For example, in the two-dimensional case, the value of a node 28 to 30 with the indices i, j in the n^{th} node layer 23, 32, 33 may be referred to as x⁽ⁿ⁾[i,j]. The arrangement of the nodes 28 to 30 of a node layer 23, 32, 33 does not have any effect on the calculations within the convolutional neural network 22 as such, since these effects are provided solely by the structure and the weights of the edges.

A convolutional layer 24 is distinguished, for example, in that the structure and the weights of the incoming edges forms a convolutional operation based on a specific number of kernels. For example, the structure and the weights of the incoming edges may be selected such that the values

$${x}_{k}^{( n )}$$

of the nodes 29 of the follower node layer 32 are ascertained as a convolution x⁽ⁿ⁾=K*x⁽ⁿ⁻¹⁾ based on the values x⁽ⁿ⁻¹⁾ of the nodes 28 of the leading node layer 23, it being possible to define the convolution * in the two-dimensional case as

$${x}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

In this, the kernel Kₖ is a d-dimensional matrix (e.g., a two-dimensional matrix) that is customarily small compared to the number of nodes 28, 29 (e.g., a 3×3 matrix or a 5×5 matrix). For example, this implies that the weights of the edges are not independent; rather, the weights are selected such that the weights generate the above convolutional equation.

In general, convolutional neural networks 22 use node layers 23, 32, 33 with a plurality of channels (e.g., owing to the use of a plurality of kernels in convolutional layers 24). In such case, the node layers may be understood as a (d+1)-dimensional matrix, with the first dimension specifying the channels. The effect of a convolutional layer 24 is then defined in a two-dimensional example as

$${x}_{b}^{( n )} [ i , j ] = {\sum }_{a} ( {K}_{a , b} * {x}^{{( n - 1 )}_{a}} ) [ i , j ] = {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ,$$

in which x⁽ⁿ⁻¹⁾ₐ corresponds to the a^{th} channel of the leading node layer 23, x⁽ⁿ⁻¹⁾_{b} corresponds to the b^{th} channel of the follower node layer 21, and K_{a,b} corresponds to one of the kernels. If a convolutional layer 24 acts on a leading node layer 23 with A channels and outputs a node layer 32 with B channels, then A·B independent d-dimensional kernels K_{a,b} exist.

In general, activation functions are used in convolutional neural networks 22. In this example embodiment, Rectified Linear Units (ReLU) are used, where R(z)=max (0, z), so the effect of the convolutional layer 24 in the two-dimensional example may be written as

$${x}_{b}^{( n )} [ i , j ] = R ( {\sum }_{a} ( {K}_{a , b} * {x}_{a}^{( n - 1 )} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ) .$$

It is also possible to use other activation functions (e.g., Exponential Linear Unit (ELU)), LeakyReLU, sigmoid functions, Tanh, or softmax.

In the represented example embodiment, the input layer 23 includes thirty-six nodes 28 that are arranged in a two-dimensional 6×6 matrix. The first hidden node layer 32 includes seventy-two nodes 29 that are arranged as two two-dimensional 6×6 matrices, with each of the two matrices being the result of a convolution of the values of the input layer 23 with a 3×3 kernel in the convolutional layer 24. The nodes 29 of the first hidden node layer 32 may be similarly arranged in a three-dimensional 2×6×6 matrix, with the first dimension corresponding to the channel dimension.

The advantage of using convolutional layers 24 is that the spatially local correlation of the input data may be exploited in that a local connection pattern between nodes of adjacent layers is created (e.g., because each node has connections to only a small range of the nodes of the preceding layer).

A pooling layer 25 is a connecting layer between a leading node layer 32 with node values x⁽ⁿ⁻¹⁾ and a follower node layer 33 with node values x⁽ⁿ⁾. A pooling layer 25 may be characterized by the structure and the weights of the edges and the activation function, with the activation function carrying out a pooling operation based on a non-linear pooling function f. For example, in the two-dimensional case, the values x⁽ⁿ⁾ of the nodes 30 of the follower node layer 33 may be calculated based on the values x⁽ⁿ⁾ of the nodes 29 of the leading node layer 32 as

$${x}_{b}^{( n )} [ i , j ] = f ( {x}_{b}^{( n - 1 )} [ {id}_{1} , {jd}_{2} ] , … , {x}_{b}^{( n - 1 )} [ ( i + 1 ) {d}_{1} - 1 , ( j + 1 ) {d}_{2} - 1 ] )$$

In other words, the number of nodes 29, 30 may be reduced by using a pooling layer 25 in that a number of d₁·d₂ adjacent nodes 29 in the leading node layer 32 is replaced by a single node 30 in the follower node layer 33, which is calculated as a function of the values of the number of adjacent nodes 29. For example, the pooling function f may be a maximal function, an averaging or the L2 norm. For example, the weights of the incoming edges may be stipulated for a pooling layer and are not modified by training.

The advantage of using a pooling layer 25 is that the number of nodes 29, 30 and the number of parameters is reduced. This results in a reduction in the necessary amount of calculation within the convolutional neural network 22 and thus in control of overfitting.

In the represented example embodiment, the pooling layer 25 is a max-pooling layer in which four adjacent nodes are replaced by just a single node having a value that is formed by the maximum of the values of the four adjacent nodes. The max-pooling is applied to each d-dimensional matrix of the previous layer; in this example embodiment, the max-pooling is applied to each of the two two-dimensional matrices, so the number of nodes reduces from seventy-two to eighteen.

In general, the last layers of a convolutional neural network 22 are fully connected layers 26. A fully connected layer 26 is a connecting layer between a leading node layer 33 and a follower node layer 27. A fully connected layer 26 is distinguished in that a plurality (e.g., all) edges between the nodes 30 of the leading node layer 33 and the nodes 31 of the follower node layer 27 are present, with it being possible for the weight of each of the edges to be individually adjusted.

In this example embodiment, the nodes 30 of the leading node layer 33 and the follower node layer 27 are both shown as two-dimensional matrices and additionally as non-contiguous nodes (e.g., represented as a row of nodes, with the number of nodes having been reduced for improved representability). This operation is also referred to as “flattening.” In this example embodiment, the number of nodes 31 in the follower node layer 27 of the fully connected layer 26 is smaller than the number of nodes 30 in the leading node layer 33 of the fully connected layer 26. In alternative embodiments, the number of nodes 31 may be the same or greater.

Further, in this example embodiment, the softmax function is applied in the fully connected layer 26. By applying the softmax function, the total of the values of all nodes 31 of the output layer 27 is one, and all values of all nodes 31 of the output layer are real numbers between 0 and 1. If the convolutional neural network 22 is used to classify input data, for example, the values of the output layer 27 may be interpreted as a probability of the input data falling into one of the different classes.

Convolutional neural networks 22 may be trained (e.g., based on the backpropagation algorithm). In order to prevent overfitting, regularization methods may be used (e.g., dropout of individual nodes 28 to 31, stochastic pooling, use of artificial data, weight decay based on the L1 or L2 norm, or maximum norm restrictions).

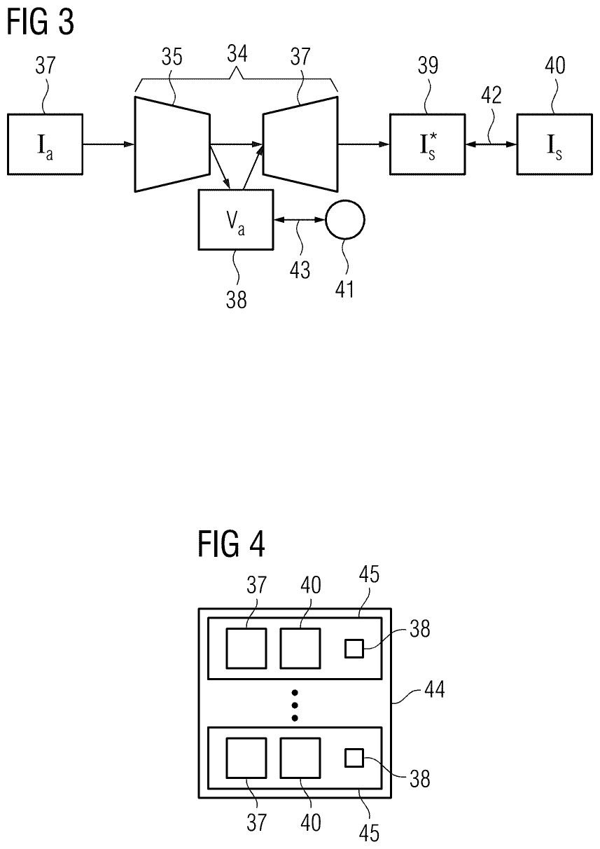

In the following example embodiments, an ascertainment function is used, which includes at least one convolutional neural network 22 and is represented in more detail in FIG. 3 abstracted in its structure. The ascertainment function 34 has an encoder 35 and a decoder 36. As input data, it receives an X-ray image (e.g., in the training procedure illustrated, a training X-ray image 37). The embedding encoder 35 ascertains therefrom a feature vector 38 in a latent space as a compact representation of the (relevant) features of the X-ray image. The decoder 36 uses the feature vector 38 in order to ascertain therefrom, as output data of the ascertainment function 34, an output image describing the scattered radiation contribution in the X-ray image that was used as input data. For example, a U-Net architecture and/or an autoencoder architecture (e.g., a variational autoencoder architecture) may be used.

Training datasets are used in the training process, which, as already mentioned, in each case include a training X-ray image 37 and a training scattered radiation image 40. The training X-ray images 37 and the training scattered radiation images 40 may be determined by simulation, but at least partially also by measurement. The training scattered radiation images 40 correspond to the basic truth.

The ascertainment function 34 is now trained first such that the output image 39 corresponds as exactly as possible to the training scattered radiation image 40, but second, contrastive learning is also used, which optimizes the distribution 41 of the feature vectors 38 in the latent space such that similar feature vectors 38 are also assigned to more similar items of scattered radiation information (e.g., more similar training scattered radiation images 40). In other words, semi-supervised learning is used with a loss function, which focuses on conformity of the output image 39 with the training scattered radiation image 40 (cf, double arrow 42), and a further loss function of the contrastive learning (e.g., a contrastive loss and/or a triplet loss), indicated by the double arrow 43. Therefore, as is also represented in FIG. 3, the feature vector 38 is branched-off as an intermediate output for this purpose.

At the end of training, an evaluation database 44 schematically represented in FIG. 4 is also compiled. For this, reference datasets 45 are formed for each training dataset, which, apart from the training X-ray image 37 and the training scattered radiation image 40, also include the feature vector 38 ascertained by the trained encoder 35 of the trained ascertainment function 34.

The trained encoder 35 as well as the evaluation database 44 are now supplied in order to ascertain for current, two-dimensional X-ray images to be evaluated (hereinafter evaluation images for short), which were recorded with an X-ray facility, the scattered radiation images, describing scattered radiation contributions contained therein, for the respective evaluation image. The method may be carried out in a control facility of the X-ray facility itself (e.g., by an integrated evaluation facility), and, at least in the case of cone beam computed tomography, then applied there to each projection image as the evaluation image.

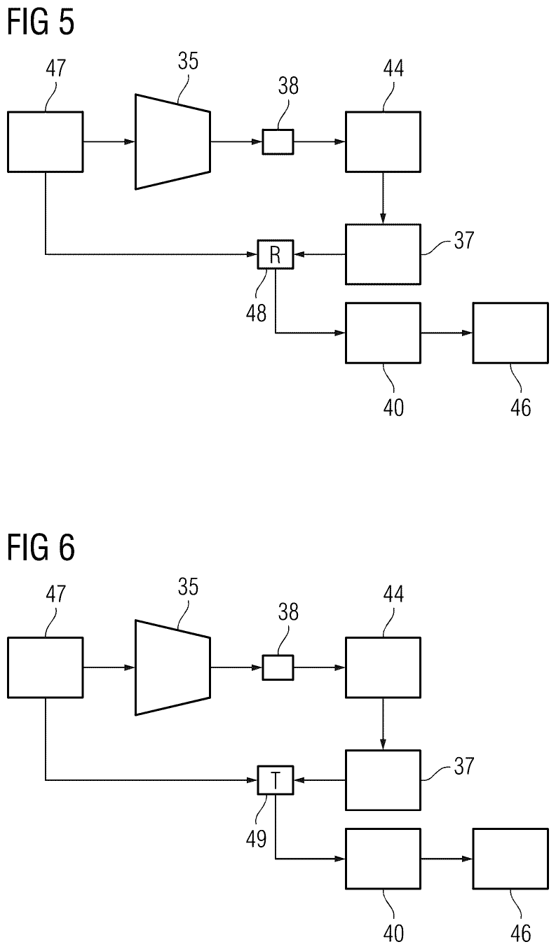

FIGS. 5 and 6 show two specific example embodiments for application of the encoder 35 and the evaluation database 44 for ascertaining a scattered radiation image 46 for an evaluation image 47. In both example embodiments, the encoder 35 is applied in a first step to the evaluation image 47 as input data in order to ascertain the feature vector 38 for the evaluation image 47. In a second act, the evaluation database 44 is then queried by the feature vector 38 in order to retrieve at least the reference dataset 45 having the feature vector 38 that most likely corresponds to the feature vector 38 of the evaluation image 47 (e.g., has a minimum distance measurement from it). The training X-ray image 37 and the training scattered radiation image 40 of the reference dataset 45 thus ascertained are retrieved.

In the example embodiment in FIG. 5, in a third act, first, the ratio 48 of the intensity of the evaluation image 47 for that of the training X-ray image 37 is formed (e.g., as the total of the image values across all image points of the evaluation image 47 divided by the total of the image values across all image points of the training X-ray image 37 of the ascertained reference dataset 45). In order to ascertain the scattered radiation image 46 for the evaluation image 47, in a fourth act, the training scattered radiation image 40 is then multiplied, therefore scaled, by the ratio 48.

In the second example embodiment in FIG. 6, in contrast to the first example embodiment in FIG. 5, it is not solely the ratio 48 of the intensities that is determined, but rather, a low-frequency transformation 49 is ascertained (e.g., such that the transformation 49 converts the training X-ray image 47 at least approximately into the output image 47). In one embodiment, the transformation 49 is derived at least partially from the feature vectors 38. In the fourth act, the transformation 49 is then applied to the training scattered radiation image 40 of the ascertained reference dataset 45 in order to ascertain the scattered radiation image 46. For example, the transformation 49 may be a deformation field and/or include a trained transformation function.

In one embodiment, both in the first example embodiment as well as in the second example embodiment, not just one reference dataset 45 with the most similar feature vector 38 may be retrieved. A plurality of reference datasets 45 may be ascertained, with it then being possible to ascertain the scattered radiation image 46 by (e.g., linear) combination of the intermediate images, scaled with the ratio 48 or transformed using the transformation 49, calculated from the training scattered radiation images 40 of the ascertained reference dataset 45. The intermediate images of the ascertained reference datasets 45 may enter the combination (e.g., with a distance measurement of the feature vector 38 of the evaluation image 47 from the feature vector 38 of the respective reference dataset 45). In one embodiment the feature vector 38 of the evaluation image 47 is represented as a linear combination of the feature vectors 38 of the ascertained reference datasets 45, with the coefficients of the linear combination being incorporated as weights in the combination of the intermediate images.

In all of the described example embodiments, the scattered radiation image 46 for the evaluation image 47 may be used for correcting the evaluation image 47 of scattered radiation contributions.

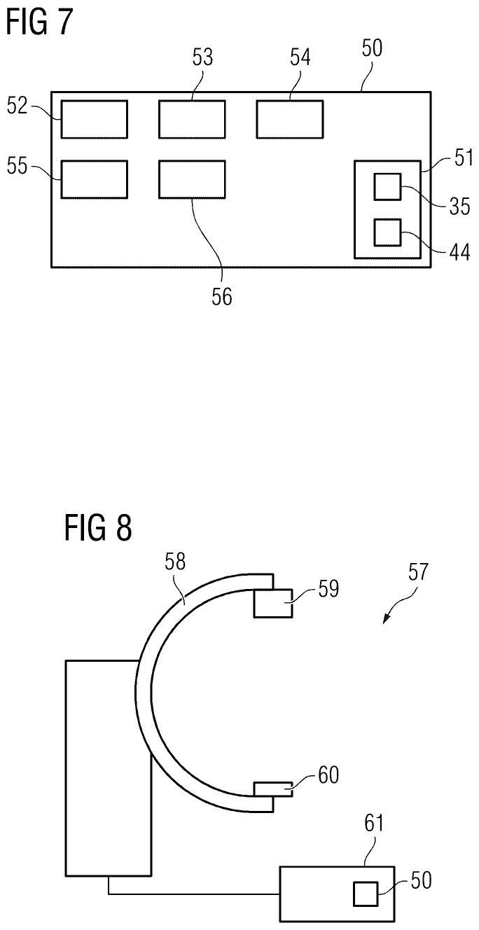

FIG. 7 shows a functional schematic diagram of an embodiment of an evaluation facility 50. The evaluation facility 50 includes a storage device 51 in which the decoder 35 and the database 44 are supplied. Optionally, the evaluation facility 50 may have a training unit 52 for carrying out the training process described in relation to FIG. 3. The evaluation facility 50 also has an application unit 53 for carrying out the first act (e.g., for applying the encoder 35 to the evaluation image 47). A first ascertainment unit 54 is provided for carrying out the second act (e.g., querying the evaluation database 44). In a second ascertainment unit 55, according to the third act and the fourth act, the scattered radiation image 46 assigned to the evaluation image 47 is ascertained, with it being possible to provide suitable subunits for the respective third act and fourth act.

Optionally, a correction unit 56 is also provided to make the described correction of the evaluation image 47 with the scattered radiation image 46.

The evaluation facility 50 is therefore configured to carry out an embodiment of a method.

FIG. 8 shows a schematic diagram of an imaging X-ray facility 57 that, in the present case, includes a C-arm 58 on which an X-ray tube assembly 59 and an X-ray detector 60 are arranged opposite one another. For example, the X-ray facility 57 may be an angiography facility (e.g., for neurovascular imaging). Two-dimensional X-ray images for diagnostics and/or monitoring interventions (e.g., therefore X-radiographs and/or fluoroscopy images) may be recorded as well as cone beam computed tomography carried out with the X-ray facility 57 in that projection images are recorded from different directions of projection. From these images, a three-dimensional image dataset is reconstructed. The X-ray facility 57 does not have an anti-scatter grid on the X-ray detector 60.

To be able to correct at least the projection images from CBCT (e.g., possibly also other two-dimensional X-ray images of scattered radiation contributions), an evaluation facility 50 of the present embodiments is integrated in a control facility 61 that controls operation of the X-ray facility 57.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for ascertaining a scattered radiation contribution to an evaluation image recorded with an X-ray facility, the method being computer-implemented and comprising:
supplying at least one encoder of a trained ascertainment function, which comprises the at least one encoder, which supplies a feature vector in a latent space for an X-ray image supplied as input data, and a decoder that ascertains an output image describing the scattered radiation contribution in the X-ray image from the feature vector;
supplying an evaluation database in which reference datasets are stored that, for each training dataset with which the ascertainment function was trained, in each case comprise at least:
a training scattered radiation image of the training dataset specifying a basic truth; and
the feature vector for the respective training dataset;
applying the at least one encoder to the evaluation image, such that a feature vector of the evaluation image is ascertained;
comparing the feature vector of the evaluation image with the feature vectors of the reference datasets, such that at least one reference dataset with the most similar feature vector is ascertained from the evaluation database; and
ascertaining a scattered radiation image for the evaluation image from the training scattered radiation images of the at least one ascertained reference dataset.

2. The method of claim 1, further comprising training the ascertainment function, training the ascertainment function comprising joint training of the at least one encoder and the decoder, the joint training comprising:
supplying training datasets that in each case comprise a training X-ray image and an associated training scattered radiation image used as a basic truth, and the ascertainment function to be trained;
training the ascertainment function using the training datasets;
storing the feature vector for each of the training datasets; and
ascertaining the reference datasets from the training datasets and the assigned, stored feature vectors.

3. The method of claim 2, wherein in the training, apart from a loss function of the supervised learning, which assesses conformity of the output image with the training scattered radiation image, a further loss function of the unsupervised learning is used that assesses the similarity of the feature vectors with respect to the similarity of the training scattered radiation images, output images, or the training scattered radiation images and the output images.

4. The method of claim 3, wherein a contrastive loss, a triplet loss, or the contrastive loss and the triplet loss are used as a further loss function.

5. The method of claim 1, further comprising ascertaining a plurality of reference datasets,
wherein the scattered radiation image is ascertained from a combination of the training scattered radiation images of the ascertained reference datasets, a combination of intermediate images derived therefrom, or a combination thereof.

6. The method of claim 5, wherein the ascertained reference datasets enter the combination with a distance measurement of the feature vector of the evaluation image from the feature vector of the respective reference dataset, the feature vector of the evaluation image is represented as a linear combination of the feature vectors of the ascertained reference datasets, or a combination thereof, and
wherein coefficients of the linear combination enter the combination of the training scattered radiation images, intermediate images, or a combination thereof as weights.

7. The method of claim 1, wherein the training X-ray image is also stored in the reference datasets, and
wherein when ascertaining the scattered radiation image for the evaluation image, an item of deviation information of the evaluation image from the training X-ray image of the at least one ascertained reference dataset is used.

8. The method of claim 7, wherein the item of deviation information comprises a ratio of intensities of the evaluation image and the training X-ray image, with which the training scattered radiation image is scaled.

9. The method of claim 7, wherein the item of deviation information comprises a transformation that links the evaluation image to the training X-ray image, is ascertained from the feature vectors of the evaluation image and the at least one ascertained reference dataset, or a combination thereof, and
wherein the transformation is applied to the training scattered radiation image.

10. The method of claim 9, wherein the transformation comprises a deformation field based on spline functions, a trained transformation function, or a combination thereof.

11. The method of claim 1, further comprising:
supplying the decoder of the trained ascertainment function;
ascertaining, by the decoder, the output image; and
using the output image for the evaluation image and the scattered radiation image for the evaluation image for a reciprocal plausibility check.

12. The method of claim 1, wherein the evaluation image is a projection image of a three-dimensional image-recording procedure in cone beam computed tomography.

13. An evaluation facility for ascertaining a scattered radiation contribution to an evaluation image recorded with an X-ray facility, the evaluation facility comprising:
a storage device configured to store:
at least one encoder of a trained ascertainment function that comprises the at least one encoder, which supplies a feature vector in a latent space for an X-ray image provided as input data, and a decoder that ascertains an output image, describing the scattered radiation contribution in the X-ray image, from the feature vector;
an evaluation database in which reference datasets are stored that, for each training dataset with which the ascertainment function was trained, in each case comprise at least:
the training scattered radiation image of the training dataset specifying a basic truth; and
the feature vector for the training dataset,
an application unit configured to apply the at least one encoder to the evaluation image, such that a feature vector of the evaluation image is ascertained;
a first ascertainment unit for comparing the feature vector of the evaluation image with the feature vectors of the reference datasets, and for ascertaining at least one reference dataset with the most similar feature vector from the evaluation database; and
a second ascertainment unit configured to ascertain a scattered radiation image for the evaluation image from the training scattered radiation images of the at least one ascertained reference dataset.

14. In a non-transitory computer-readable storage medium that stores instructions executable by one or more processors to ascertain a scattered radiation contribution to an evaluation image recorded with an X-ray facility, the instructions comprising:
supplying at least one encoder of a trained ascertainment function, which comprises the at least one encoder, which supplies a feature vector in a latent space for an X-ray image supplied as input data, and a decoder that ascertains an output image describing the scattered radiation contribution in the X-ray image from the feature vector;
supplying an evaluation database in which reference datasets are stored that, for each training dataset with which the ascertainment function was trained, in each case comprise at least:
a training scattered radiation image of the training dataset specifying a basic truth; and
the feature vector for the respective training dataset;
applying the at least one encoder to the evaluation image, such that a feature vector of the evaluation image is ascertained;
comparing the feature vector of the evaluation image with the feature vectors of the reference datasets, such that at least one reference dataset with the most similar feature vector is ascertained from the evaluation database; and
ascertaining a scattered radiation image for the evaluation image from the training scattered radiation images of the at least one ascertained reference dataset.

15. The non-transitory computer-readable storage medium of claim 14, wherein the instructions further comprise training the ascertainment function, training the ascertainment function comprising joint training of the at least one encoder and the decoder, the joint training comprising:
supplying training datasets that in each case comprise a training X-ray image and an associated training scattered radiation image used as a basic truth, and the ascertainment function to be trained;
training the ascertainment function using the training datasets;
storing the feature vector for each of the training datasets; and
ascertaining the reference datasets from the training datasets and the assigned, stored feature vectors.

16. The non-transitory computer-readable storage medium of claim 15, wherein in the training, apart from a loss function of the supervised learning, which assesses conformity of the output image with the training scattered radiation image, a further loss function of the unsupervised learning is used that assesses the similarity of the feature vectors with respect to the similarity of the training scattered radiation images, output images, or the training scattered radiation images and the output images.

17. The non-transitory computer-readable storage medium of claim 16, wherein a contrastive loss, a triplet loss, or the contrastive loss and the triplet loss are used as a further loss function.
